(19) United States
(12) Patent Application Publication
(10) Pub. No.: US 2024/0335141 A1
(43) Pub. Date: Oct. 10, 2024

(54) SYSTEMS AND METHODS FOR MOBILE SPEECH HEARING OPTIMIZATION

(71) Applicant: Linus Health, Inc., Boston, MA (US)

(72) Inventors: Russell Banks, Boston, MA (US); Joyce Gomes-Osman, Boston, MA (US); Alvaro Pascual-Leone, Boston, MA (US); Isaiah Morrow, Waltham, MA (US); Ali Jannati, Boston, MA (US); Barry R. Greene, Dublin (IE)

(21) Appl. No.: 18/627,964

(22) Filed: Apr. 5, 2024

Related U.S. Application Data
(60) Provisional application No. 63/494,533, filed on Apr. 6, 2023.

(51) Int. Cl. A61B 5/12; A61B 5/00

(52) U.S. Cl. CPC A61B 5/123; A61B 5/741; A61B 5/743; A61B 5/7435; A61B 5/7475

(57) ABSTRACT

Systems and methods for speech hearing screening are described. A method of speech hearing screening comprises receiving an input from a user to indicate a screening is beginning; playing a made-up word on an external speaker at a volume for the user; displaying a series of words to the user to match to the made-up word; and receiving a selection of one word of the series of words from the user. A system of speech-hearing screening comprises a tablet device; a computing node configured to perform a method comprising receiving an input from a user to indicate a screening is beginning; playing a first made-up word on an external speaker at a first volume for the user; displaying a first series of words to the user to match to the first made-up word; and receiving a selection of one word of the series of words from the user.

### RELATED APPLICATION(S)

This application claims the benefit of priority to U.S. Provisional Application No. 63/494,533, filed Apr. 6, 2023, the entirety of which is incorporated herein by reference.

### TECHNICAL FIELD

The invention relates generally to hearing tests, and, in particular, to systems and methods for optimizing speech hearing using a mobile application.

### BACKGROUND

Currently it is difficult to determine whether users of a mobile health platform have hearing issues, limiting the ability of the mobile health platform to evaluate patients. Mobile health platforms issue instructions and key components of health assessments, including cognitive assessments, via external speakers. There is a need for an interactive speech-hearing screener to help disassociate hearing and cognitive issues in users, and to give ample opportunities for platform users to access instructions and activities. Further, by using the interactive speech-hearing screener, the platform volume will automatically be set at a level that users have indicated they can hear and understand stimuli provided by a device's external speakers. As the screener test is fully contained by the platform and uses only a mobile device's built-in speakers, it does not rely on external devices.

### SUMMARY

According to certain aspects of the present disclosure, systems and methods for optimizing speech hearing using a mobile application are disclosed.

In one embodiment, a method for speech hearing screening comprises receiving an input from a user to indicate a screening is beginning; playing a single made-up word on an external speaker at a first volume for the user; displaying a first series of words to the user to match to the first made-up word; and receiving a selection of one word of the series of words from the user.

In another embodiment, a system for speech hearing screening comprises a tablet device with external speakers; a computing node comprising a computer readable storage medium having program instructions embodied therein, the program instructions executable by a processor of the computing node to cause the processor to perform a method comprising receiving an input from a user to indicate a screening is beginning; playing a first made-up word on an external speaker at a first volume for the user; displaying a first series of words to the user to match to the first made-up word; and receiving a selection of one word of the series of words from the user.

In an alternate embodiment, a computer program product for screening speech hearing, the computer program product comprising a non-transitory computer readable storage medium having program instructions embodied therewith, the program instructions executable by a processor to cause the processor to perform a method comprising receiving an input from a user to indicate a screening is beginning; playing a first made-up word on an external speaker at a first volume for the user; displaying a first series of words to the user to match to the first made-up word; and receiving a selection of one word of the series of words from the user.

### BRIEF DESCRIPTION OF THE DRAWINGS

The accompanying drawings, which are incorporated into and constitute a part of this specification, illustrate various exemplary embodiments and together with the description, serve to explain the principles of the disclosed embodiments.

FIG. 1 is an exemplary workflow of a speech hearing test on a mobile device, according to embodiments of the present disclosure.

FIG. 2A is an illustration of the setup used to measure the decibel loudness of a mobile device spaced in relation to a microphone, and used to determine the loudness of the mobile device according to embodiments of the present disclosure.

FIG. 2B is a graph displaying intensity level in dB at respective volumes, according to embodiments of the present disclosure.

FIG. 3A is a graph displaying volume by pure tone average, according to embodiments of the present disclosure.

FIG. 3B is a graph displaying volume by speech recognition threshold, according to embodiments of the present disclosure.

FIG. 4 is an exemplary computing node, according to embodiments of the present disclosure.

### DETAILED DESCRIPTION

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

Several drawbacks exist in current hearing screening mobile tests. First, most tests are “pure tone” screeners, presenting a single beep at a specific frequency and loudness requiring the user to indicate that they can hear it. This is done for a range of standardized frequencies and patterned after clinical audiometry. The results of this test are limited, and an individual's speech hearing can only be inferred based on the tones presented. Current solutions mimic audiometry but are not, in fact, comparable or equivalent as they do not require calibration or provide an auditory reference. By mimicking audiometry with pure tones, these solutions do not assess a person's ability to hear speech, and thus cannot make any truly meaningful claims about the subject's speech hearing ability. These also become unnecessarily long screenings, as the test needs to be repeated using calibrated machinery.

Additionally, current solutions require the use of in-ear headphones, presenting issues with calibration, correct lateralization, sanitation, and accessibility. Further, headphones present the need for additional maintenance on the headphone equipment in case of malfunction. Calibration is also required for some of the currently available solutions, putting additional responsibility on test administration staff.

Some known hearing screeners are well designed, but none meet the needs of platform users. All require external headphone devices, administrator training, and results do not inform or enhance performance on the mobile app platform assessments. Embodiments of the hearing screening test described in the present disclosure provide insight into the user's ability to hear speech delivered by the mobile device, in preparation for receiving verbal instructions from the device speakers and performing cognitive screening on the mobile platform. Known screening tests only give an indication as to the frequencies that users can or cannot hear, without specifically addressing the user's speech hearing ability.

Embodiments of the present disclosure are speech hearing and platform optimization tools. By screening the volume level that a user can hear, it is possible to increase the certainty that users are able to hear instructions given by the mobile application and device speakers. For tasks requiring the verbal repetition of auditory stimuli, it is anticipated that overall user performance will increase as they will have more access to stimuli presented at a louder level established by each individual user.

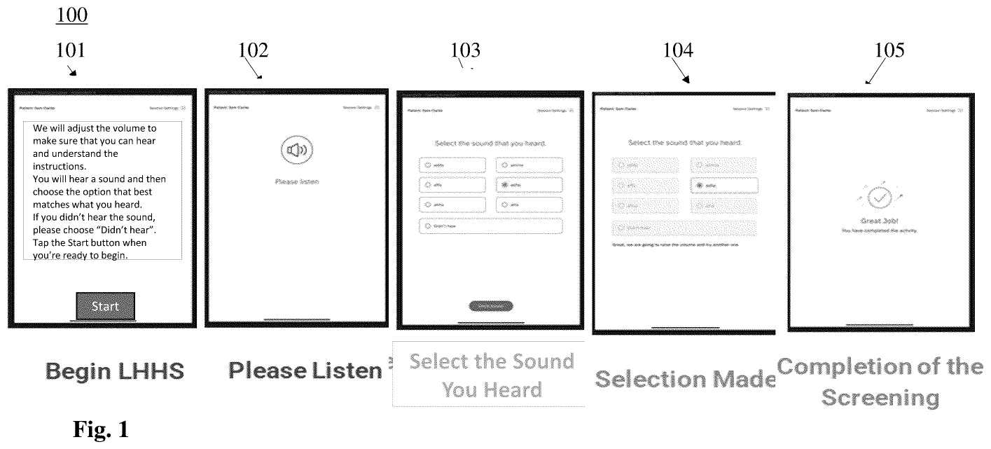

FIG. 1 is an exemplary workflow 100 of a speech hearing test on a mobile device, according to embodiments of the present disclosure. The workflow 100 can be used on a tablet, smartphone, or any other suitable computing device. Upon opening an application, a user is presented with the screen as shown in step 101. An explanatory text will instruct the user on how to proceed with the screening. In step 101, the text tells the user that the volume will be adjusted to make sure that the user can hear and understand the instructions. The user will hear a sound and then choose the option that best matches what was heard. If the user does not hear the sound, the “Didn't Hear” option can be selected. When ready, the user taps a start button on the bottom of the screen.

Once the workflow 100 has begun by the user selecting the start button, the screen will display a “Please Listen” signal to indicate that a noise is being made, as shown in step 102. The volume will be adjusted throughout the completion of the screening to ensure that the user can hear and understand the instructions that are presented as parted as part of the cognitive screening that can take place following the hearing screening. Users will hear a short, made-up vowel-consonant-vowel (VCV) word presented by the mobile device's external speakers. The VCV words were chosen for their similarity to English word structure, but are not real English words and thus do not interfere with verbal memory tests often implemented in cognitive testing. Users are then shown a list of similar word options presented on the screen, as in step 103, and asked to choose the word that best matches what the user heard. If the user didn't hear the sound, the user can select the “Didn't hear” option. If the correct word is selected, the same process will be repeated with different words. The user is asked to do the same task for several different made-up words until the patient correctly identifies three words in a row. Each time a word is incorrectly identified, the volume is increased by one volume button increment on the mobile device, until 100% of the device volume is reached or three words are correctly identified in a row. Once three words are correctly identified, the volume of the device is set and the cognitive screening is delivered at that volume.

If a word is incorrectly identified, instructions will be displayed and read via the device's external speakers. For example, the message “Great, we are going to raise the volume and try another one” may be displayed, as shown in step 104. The user will understand that the volume of the device will be raised incrementally each time an incorrect response is given.

Upon the completion of the hearing screening, a message is displayed as shown in step 105 to indicate the end of the activity. The screening is complete when the three words are correctly identified, or the volume reaches 100%.

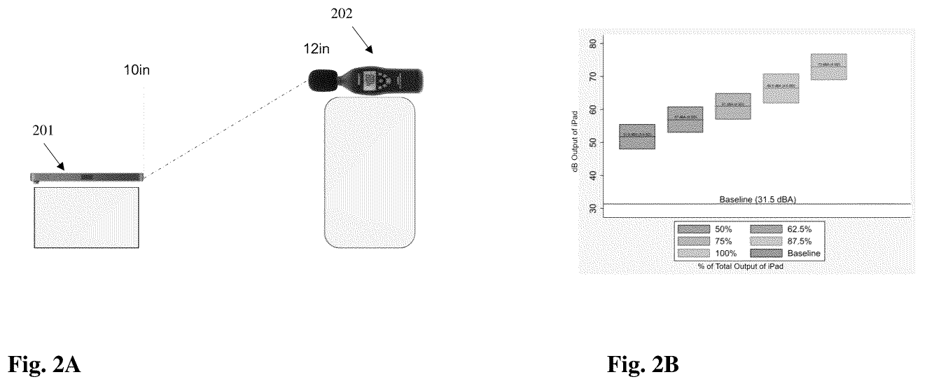

FIG. 2A is an illustration of a mobile device spaced in relation to a recorder, according to embodiments of the present disclosure. The exemplary setup of tablet in a preliminary study of the tablet volumes 201 in relation to recorder 202 recorded volumes at 50, 62.5, 75, 87.5, and 100% of the maximum tablet 201 and reported the output at each volume in decibels (dB). Thus, the recording information can be relevant to loudness levels (in dB) of the tablet at different percentages of the volume output of the tablet. The dB output of the tablet at each of the volumes 50-100% deliver stimulus at loudness appropriate for normal (50%), mildly impaired (62.5%) and severely impaired hearing (75-100%).

FIG. 2B is a graph displaying intensity level in dB at respective volumes, according to embodiments of the present disclosure. The intensity level in dB at each of the respective tablet volumes 50% and above.

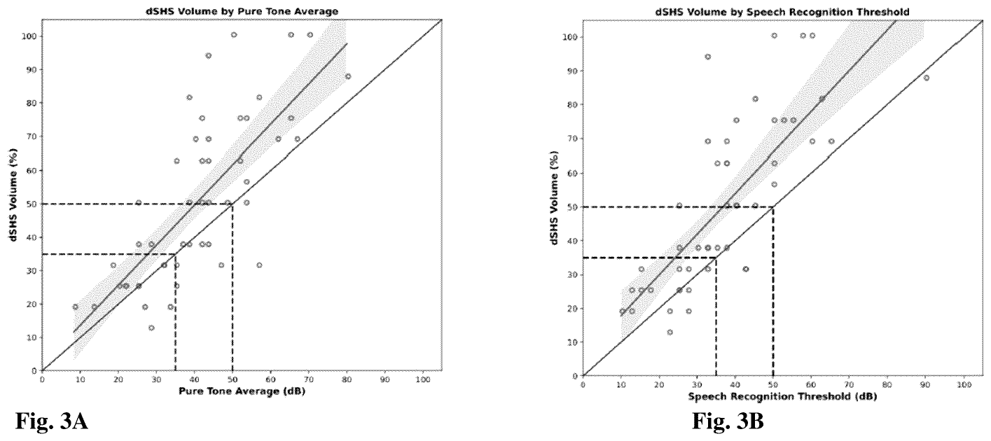

FIG. 3A is a graph displaying dSHS volume by Pure Tone Average, according to embodiments of the present disclosure. In the exemplary graph, resultant tablet dSHS volume percentages are shown, comparing the volume in decibels (dB) as obtained by workflow 100 with the Pure Tone Average (PTA) test. The PTA was determined by audiometry administered by a licensed hearing specialist and is the average of an individual's hearing ability in dB at 500, 1,000, and 2,000 Hz frequencies (the frequencies most important for understanding speech). The PTA is the clinical standard for objectively quantifying an individual's ability to hear speech.

FIG. 3B is a graph displaying dSHS volume by speech recognition threshold, according to embodiments of the present disclosure. The exemplary graph shows tablet dB dSHS volume percentages are shown, corresponding to the resulting speech recognition thresholds as obtained by the method of workflow 100.

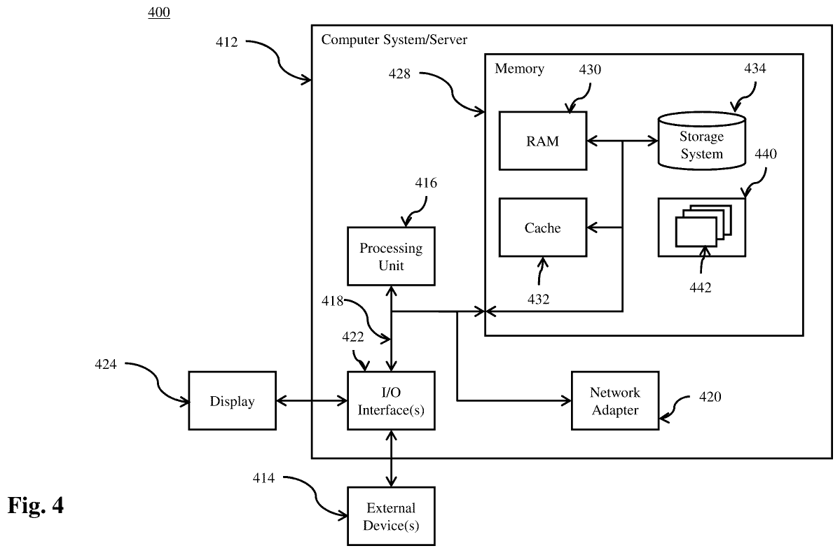

Referring now to FIG. 4, a schematic of an example of a computing node is shown. Computing node 410 is only one example of a suitable computing node and is not intended to suggest any limitation as to the scope of use or functionality of embodiments described herein. Regardless, computing node 410 is capable of being implemented and/or performing any of the functionality set forth hereinabove.

In computing node 410 there is a computer system/server 412, which is operational with numerous other general purpose or special purpose computing system environments or configurations. Examples of well-known computing systems, environments, and/or configurations that may be suitable for use with computer system/server 412 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed computing environments that include any of the above systems or devices, and the like.

Computer system/server 412 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 412 may be practiced in distributed computing environments where tasks are performed by remote processing devices that are linked through a communications network. In a distributed computing environment, program modules may be located in both local and remote computer system storage media including memory storage devices.

As shown in FIG. 4, computer system/server 412 in computing node 410 is shown in the form of a general-purpose computing device. The components of computer system/server 412 may include, but are not limited to, one or more processors or processing units 416, a system memory 428, and a bus 418 that couples various system components including system memory 428 to processor 416.

Bus 418 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 412 typically includes a variety of computer system readable media. Such media may be any available media that is accessible by computer system/server 412, and it includes both volatile and non-volatile media, removable and non-removable media.

System memory 428 can include computer system readable media in the form of volatile memory, such as random access memory (RAM) 430 and/or cache memory 432. Computer system/server 412 may further include other removable/non-removable, volatile/non-volatile computer system storage media. By way of example only, storage system 434 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 418 by one or more data media interfaces. As will be further depicted and described below, memory 428 may include at least one program product having a set (e.g., at least one) of program modules that are configured to carry out the functions of embodiments of the disclosure.

Program/utility 440, having a set (at least one) of program modules 442, may be stored in memory 428 by way of example, and not limitation, as well as an operating system, one or more application programs, other program modules, and program data. Each of the operating system, one or more application programs, other program modules, and program data or some combination thereof, may include an implementation of a networking environment. Program modules 442 generally carry out the functions and/or methodologies of embodiments as described herein.

Computer system/server 412 may also communicate with one or more external devices 414 such as a keyboard, a pointing device, a display 424, etc.; one or more devices that enable a user to interact with computer system/server 412; and/or any devices (e.g., network card, modem, etc.) that enable computer system/server 412 to communicate with one or more other computing devices. Such communication can occur via Input/Output (I/O) interfaces 422. Still yet, computer system/server 412 can communicate with one or more networks such as a local area network (LAN), a general wide area network (WAN), and/or a public network (e.g., the Internet) via network adapter 420. As depicted, network adapter 420 communicates with the other components of computer system/server 412 via bus 418. It should be understood that although not shown, other hardware and/or software components could be used in conjunction with computer system/server 412. Examples, include, but are not limited to: microcode, device drivers, redundant processing units, external disk drive arrays, RAID systems, tape drives, and data archival storage systems, etc.

In various embodiments, a learning system is provided. In some embodiments, a feature vector is provided to a learning system. Based on the input features, the learning system generates one or more outputs. In some embodiments, the output of the learning system is a feature vector. In some embodiments, the learning system comprises an SVM. In other embodiments, the learning system comprises an artificial neural network. In some embodiments, the learning system is pre-trained using training data. In some embodiments training data is retrospective data. In some embodiments, the retrospective data is stored in a data store. In some embodiments, the learning system may be additionally trained through manual curation of previously generated outputs.

In some embodiments, the learning system, is a trained classifier. In some embodiments, the trained classifier is a random decision forest. However, it will be appreciated that a variety of other classifiers are suitable for use according to the present disclosure, including linear classifiers, support vector machines (SVM), or neural networks such as recurrent neural networks (RNN).

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed:

1. A method of screening speech hearing, the method comprising:
receiving an input from a user to indicate a screening is beginning;
playing a first made-up word on an external speaker at a first volume for the user;
displaying a first series of words to the user to match to the first made-up word; and
receiving a selection of one word of the series of words from the user.

2. The method of claim 1, further comprising:
determining that the one word selected by the user matches the made-up word;
playing a second made-up word on the external speaker at the first volume;
displaying a second series of words to the user to match to the second made-up word; and
receiving a second selection of one word of the second series of words from the user.

3. The method of claim 2, further comprising:
determining that the second selection of one word by the user matches the second made-up word;
playing a third made-up word on the external speaker at the first volume;
displaying a third series of words to the user to match to the third made-up word;
receiving a third selection of one word of the third series of words from the user;
determining that the third selection of one word by the user matches the third made-up word; and
setting a default volume of the external speaker to the first volume; and
indicating to the user that the screening is complete.

4. The method of claim 1, further comprising:
determining that the one word selected by the user is different from the first made-up word;
playing a second made-up word on the external speaker at a second volume;
displaying a second series of words to the user to match to the second made-up word; and
receiving a second selection of one word of the second series of words from the user.

5. The method of claim 4, wherein the second volume is an increment above the first volume.

6. The method of claim 4, further comprising:
increasing the external speaker to a maximum volume upon a subsequent incorrect match between a played word and a selected word; and
indicating to the user that the screening is complete.

7. The method of claim 1, wherein the made-up word comprises a vowel-consonant-vowel word.

8. The method of claim 7, wherein the made-up word is generated from a word bank.

9. The method of claim 7, wherein the made-up word is randomly generated.

10. A system for screening speech hearing, the system comprising:
a tablet device with external speakers;
a computing node comprising a computer readable storage medium having program instructions embodied therein, the program instructions executable by a processor of the computing node to cause the processor to perform a method comprising:
receiving an input from a user to indicate a screening is beginning;
playing a first made-up word on an external speaker at a first volume for the user;
displaying a first series of words to the user to match to the first made-up word; and
receiving a selection of one word of the series of words from the user.

11. The system of claim 10, further comprising:
determining that the one word selected by the user matches the made-up word;
playing a second made-up word on the external speaker at the first volume;
displaying a second series of words to the user to match to the second made-up word; and
receiving a second selection of one word of the second series of words from the user.

12. The system of claim 11, further comprising:
determining that the second selection of one word by the user matches the second made-up word;
playing a third made-up word on the external speaker at the first volume;
displaying a third series of words to the user to match to the third made-up word;
receiving a third selection of one word of the third series of words from the user;
determining that the third selection of one word by the user matches the third made-up word; and
setting a default volume of the external speaker to the first volume; and
indicating to the user that the screening is complete.

13. The system of claim 10, further comprising:
determining that the one word selected by the user is different from the first made-up word;
playing a second made-up word on the external speaker at a second volume;
displaying a second series of words to the user to match to the second made-up word; and
receiving a second selection of one word of the second series of words from the user.

14. The system of claim 13, wherein the second volume is an increment above the first volume.

15. The system of claim 13, further comprising:
increasing the external speaker to a maximum volume upon a subsequent incorrect match between a played word and a selected word; and
indicating to the user that the screening is complete.

16. The system of claim 10, wherein the made-up word comprises a vowel-consonant-vowel word.

17. The system of claim 16, wherein the made-up word is generated from a word bank.

18. The system of claim 16, wherein the made-up word is randomly generated.

19. A computer program product for screening speech hearing, the computer program product comprising a non-transitory computer readable storage medium having program instructions embodied therewith, the program instructions executable by a processor to cause the processor to perform a method comprising:
receiving an input from a user to indicate a screening is beginning;
playing a first made-up word on an external speaker at a first volume for the user;
displaying a first series of words to the user to match to the first made-up word; and
receiving a selection of one word of the series of words from the user.

20. The computer program product of claim 19, further comprising:
determining that the one word selected by the user matches the made-up word;
playing a second made-up word on the external speaker at the first volume;
displaying a second series of words to the user to match to the second made-up word; and
receiving a second selection of one word of the second series of words from the user.
